(19) United States
(12) Patent Application Publication
(10) Pub. No.: US 2023/0420126 A1
(43) Pub. Date: Dec. 28, 2023

(54) BLOODSTREAM INFECTION PREDICTING SYSTEM AND METHOD THEREOF

(71) Applicants: Taichung Veterans General Hospital, Taichung City (TW); Tunghai University, Taichung (TW)

(72) Inventors: Chieh-Liang WU, Taichung City (TW); Po-Yu LIU, Taichung City (TW); Kai-Chih PAI, Taichung (TW); Lai-Shiun LAI, Taichung City (TW); Min-Shian WANG, Taichung City (TW); Ruey-Kai SHEU, Taichung (TW); Lun-Chi CHEN, Taichung (TW)

(21) Appl. No.: 17/847,475

(22) Filed: Jun. 23, 2022

(51) Int. Cl. G16H 50/20; G16H 50/30

(52) U.S. Cl. CPC G16H 50/20; G16H 50/30

(57) ABSTRACT

A bloodstream infection predicting system and a method thereof are proposed. The memory unit stores a plurality of historical medical data, the real-time data to be tested and a machine learning algorithm. The processor is configured to implement a bloodstream infection predicting method. The bloodstream infection predicting method includes reading the historical medical data from the memory unit, training the historical medical data to generate a bloodstream infection prediction model, reading the real-time data to be tested of the patient from the memory unit, and inputting the real-time data to be tested into the bloodstream infection prediction model to generate the bloodstream infection risk probability. The real-time data to be tested includes an intensive care unit detecting data and a blood inspection data of the patient. The intensive care unit detecting data and the blood inspection data are detected during a feature window time interval.

### BACKGROUND

### Technical Field

The present disclosure relates to an infection predicting system and a method thereof. More particularly, the present disclosure relates to a bloodstream infection predicting system and a method thereof.

### Description of Related Art

Bloodstream infection is a common serious disease in the Intensive Care Unit (ICU), but it is not easy to be diagnosed immediately. The diagnosis of the bloodstream infection is identified through a blood culture, and a medical staff gives a medical treatment to the bloodstream infection patient after the blood culture is completed. The bloodstream infection is a main cause of death of the critical patients and the risk of death of the blood infection patient is extremely high. Therefore, giving the treatment to the bloodstream infection patient after the blood culture may miss the optimal treatment time of the patient.

In summary, there is still a lack of a bloodstream infection predicting system and a method thereof monitoring the health of the patient in the ICU immediately, which are indeed highly anticipated by the public and become the goal and the direction of relevant industry efforts.

### SUMMARY

The purpose of the present disclosure is providing a bloodstream infection predicting system and a method thereof, to predict a bloodstream infection risk probability in a specific time interval by collecting a real-time data to be tested of a patient.

According to one aspect of the present disclosure, a bloodstream infection predicting system is configured to predict a bloodstream infection risk probability according to a real-time data to be tested of a patient. The bloodstream infection predicting system includes a memory unit and a processor. The memory unit stores a plurality of historical medical data, the real-time data to be tested and a machine learning algorithm. The processor is signally connected to the memory unit, and configured to implement a bloodstream infection predicting method. The bloodstream infection predicting method includes performing a first data reading step, a model training step, a second data reading step and a risk predicting step. The first data reading step is performed to read the historical medical data from the memory unit. The model training step is performed to train the historical medical data according to the machine learning algorithm to generate a bloodstream infection prediction model. The second data reading step is performed to read the real-time data to be tested of the patient from the memory unit. The risk predicting step is performed to input the real-time data to be tested into the bloodstream infection prediction model to generate the bloodstream infection risk probability. The real-time data to be tested includes an intensive care unit detecting data and a blood inspection data of the patient. The intensive care unit detecting data and the blood inspection data are detected during a feature window time interval.

Therefore, the bloodstream infection predicting system of the present disclosure can predict the bloodstream infection risk probability in a specific time interval, thereby giving a medical treatment to the patient immediately.

According to one embodiment, the memory unit stores a predetermined number and a predetermined lower limit number. Each of the historical medical data comprises a plurality of feature data. The bloodstream infection predicting method further includes performing a data pre-processing step. The data pre-processing step includes configuring the processor to calculate an average value of each of the feature data and configuring the processor to judge whether a number of the feature data of each of the historical medical data is less than or equal to the predetermined lower limit number. In response to determining that the number of the feature data of one of the historical medical data is less than or equal to the predetermined lower limit number, the processor removes the one of the historical medical data. In response to determining that the number of the feature data of the one of the historical medical data is greater than the predetermined lower limit number and less than the predetermined number, the processor fills the average values corresponding to a missing part of the feature data of the one of the historical medical data in the one of the historical medical data according to an interpolation process to let the number of the feature data of the one of the historical medical data be equal to the predetermined number.

According to one embodiment, the intensive care unit detecting data includes a temperature, a respiration rate, a pulse rate, a pulse pressure, a Systolic Blood Pressure (SBP), a Diastolic Blood Pressure (DBP), a Glasgow Coma Scale (GCS) and a catheter insertion time data.

According to one embodiment, the blood inspection data includes a lactate, an arterial blood gas_pH and a HCO₃-A value.

According to one embodiment, the machine learning algorithm is one of a logistic regression, a Support Vector Machine (SVM), a MultiLayer Perceptron (MLP), a random forest and an eXtreme Gradient Boosting (XGBoost).

According to another aspect of the present disclosure, a bloodstream infection predicting method is configured to predict a bloodstream infection risk probability according to a real-time data to be tested of a patient. The bloodstream infection predicting method includes performing a first data reading step, a model training step, a second data reading step and a risk predicting step. The first data reading step is performed to configure a processor to read a plurality of historical medical data from a memory unit. The model training step is performed to configure the processor to train the historical medical data according to a machine learning algorithm to generate a bloodstream infection prediction model. The second data reading step is performed to configure the processor to read the real-time data to be tested of the patient from the memory unit. The risk predicting step is performed to configure the processor to input the real-time data to be tested into the bloodstream infection prediction model to generate the bloodstream infection risk probability. The real-time data to be tested includes an intensive care unit detecting data and a blood inspection data of the patient. The intensive care unit detecting data and the blood inspection data are detected during a feature window time interval.

Therefore, the bloodstream infection predicting method of the present disclosure can predict the bloodstream infection risk probability in a specific time interval, thereby giving a medical treatment to the patient immediately.

According to one embodiment, the memory unit stores a predetermined number and a predetermined lower limit number. Each of the historical medical data includes a plurality of feature data. The bloodstream infection predicting method further includes performing a data pre-processing step. The data pre-processing step includes configuring the processor to calculate an average value of each of the feature data and configuring the processor to judge whether a number of the feature data of each of the historical medical data is less than or equal to the predetermined lower limit number. In response to determining that the number of the feature data of one of the historical medical data is less than or equal to the predetermined lower limit number, the processor removes the one of the historical medical data. In response to determining that the number of the feature data of the one of the historical medical data is greater than the predetermined lower limit number and less than the predetermined number, the processor fills the average values corresponding to a missing part of the feature data of the one of the historical medical data in the one of the historical medical data according to an interpolation process to let the number of the feature data of the one of the historical medical data be equal to the predetermined number.

According to one embodiment, the intensive care unit detecting data includes a temperature, a respiration rate, a pulse rate, a pulse pressure, a Systolic Blood Pressure (SBP), a Diastolic Blood Pressure (DBP), a Glasgow Coma Scale (GCS) and a catheter insertion time data.

According to one embodiment, the blood inspection data includes a lactate, an arterial blood gas_pH and a HCO₃-A value.

According to one embodiment, the machine learning algorithm is one of a logistic regression, a Support Vector Machine (SVM), a MultiLayer Perceptron (MLP), a random forest and an eXtreme Gradient Boosting (XGBoost).

### BRIEF DESCRIPTION OF THE DRAWINGS

FIG. 1 shows a block diagram of a bloodstream infection predicting system according to a first embodiment of the present disclosure.

FIG. 2 shows a schematic view of a real-time data to be tested of the bloodstream infection predicting system of FIG. 1.

FIG. 3 shows a flow chart of a bloodstream infection predicting method according to a second embodiment of the present disclosure.

FIG. 4 shows a schematic view of a feature window time interval of the bloodstream infection predicting method of FIG. 3.

### DETAILED DESCRIPTION

The embodiment will be described with the drawings. For clarity, some practical details will be described below. However, it should be noted that the present disclosure should not be limited by the practical details, that is, in some embodiment, the practical details is unnecessary. In addition, for simplifying the drawings, some conventional structures and elements will be simply illustrated, and repeated elements may be represented by the same labels.

It will be understood that when an element (or device) is referred to as be “connected to” another element, it can be directly connected to the other element, or it can be indirectly connected to the other element, that is, intervening elements may be present. In contrast, when an element is referred to as be “directly connected to” another element, there are no intervening elements present. Consequently, a first element or component discussed below could be termed a second element or component.

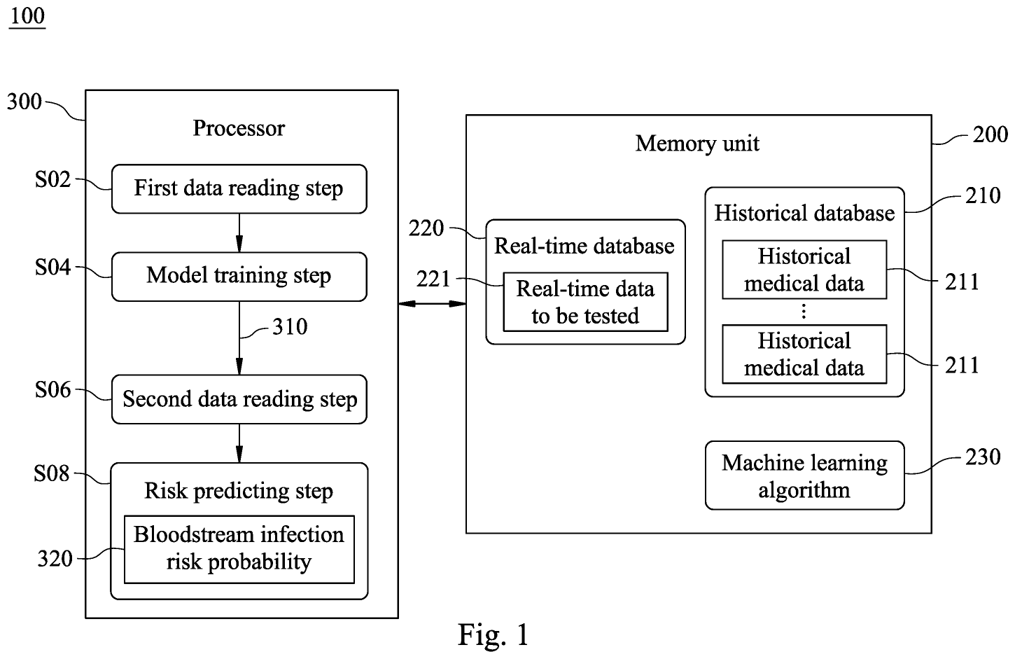

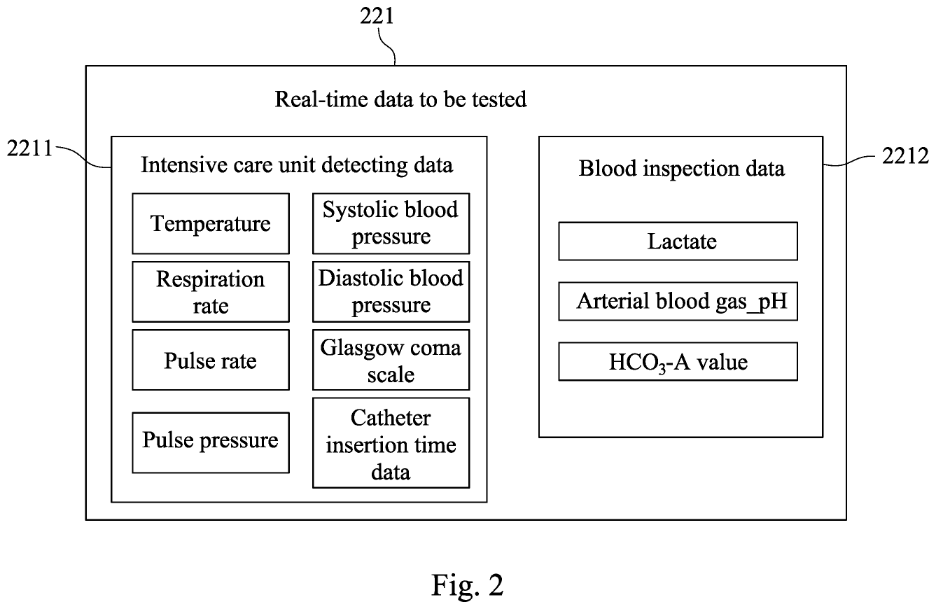

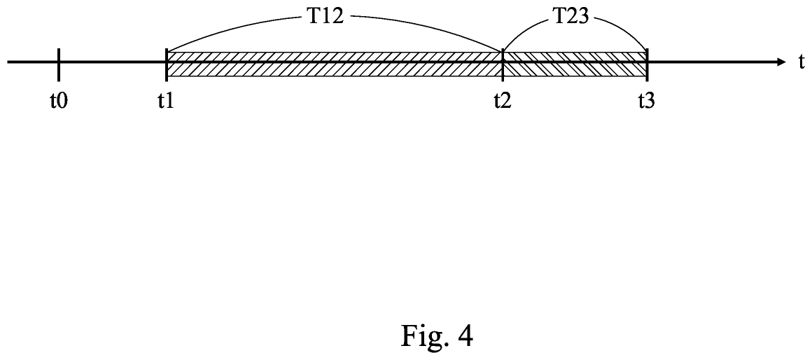

Please refer to FIG. 1 and FIG. 2. FIG. 1 shows a block diagram of a bloodstream infection predicting system 100 according to a first embodiment of the present disclosure. FIG. 2 shows a schematic view of a real-time data to be tested 221 of the bloodstream infection predicting system 100 of FIG. 1. The bloodstream infection predicting system 100 is configured to predict a bloodstream infection risk probability 320 according to the real-time data to be tested 221 of a patient. The bloodstream infection predicting system 100 includes a memory unit 200 and a processor 300. The memory unit 200 stores a plurality of historical medical data 211, the real-time data to be tested 221 and a machine learning algorithm 230. The real-time data to be tested 221 includes an intensive care unit (ICU) detecting data 2211 and a blood inspection data 2212 of the patient. The ICU detecting data 2211 and the blood inspection data 2212 are detected during a feature window time interval T12 (as shown in FIG. 4).

In detail, the memory unit 200 stores a historical database 210, a real-time database 220 and the machine learning algorithm 230. The memory unit 200 can be a memory or other data storing element. The historical database 210 includes the historical medical data 211. Each of the historical medical data 211 includes a historical feature data of a patient, who has admitted to an ICU. The real-time database 220 includes the ICU detecting data 2211 and the blood inspection data 2212 of a patient to be tested, and the ICU detecting data 2211 and the blood inspection data 2212 are detected during the feature window time interval T12. In the embodiment of FIG. 1, the real-time database 220 stores the real-time data to be tested 221 of the patient, who is admitted to the ICU, in 72 hours (i.e., the feature window time interval T12).

In detail, the real-time data to be tested 221 can include the ICU detecting data 2211, the blood inspection data 2212 and other feature data of a patient. The ICU detecting data 2211, the blood inspection data 2212 and other feature data are detected in the ICU. The ICU detecting data 2211 can include a temperature, a respiration rate, a pulse rate, a pulse pressure, a Systolic Blood Pressure (SBP), a Diastolic Blood Pressure (DBP), a Glasgow Coma Scale (GCS) and a catheter insertion time data. The catheter insertion time data can include a SwanGanze insertion time, an Endotracheal (ENDO) tube insertion time, a foley insertion time, a Central Venous Catheter (CVC) insertion time, a central venous pressure catheter insertion time, a double lumen insertion time, a hickman catheter insertion time, a Peripherally Inserted Central Catheters (PICC) insertion time and a Port A insertion time, but the present disclosure is not limited thereto.

The blood inspection data 2212 can include a lactate, an arterial blood gas_pH, HCO₃-A value, a White Blood Cell count (WBC-min), a Blood Urea Nitrogen (BUN), an AlkalinePhosphatase (ALKP), a Hemoglobin (Hb), a Sodium(K), a creatinine and a ProthrombinTime-C. In the embodiment of FIG. 1, the other feature data can include an Acute Physiology and Chronic Health Evaluation (APACHE) II score, but the present disclosure is not limited thereto.

The processor 300 can be a microprocessor, a Central Processing Unit (CPU) or other electronic computing processor, but the present disclosure is not limited thereto. The processor 300 is signally connected to the memory unit 200, and configured to implement a first data reading step S02, a model training step S04, a second data reading step S06 and a risk predicting step S08. The first data reading step S02 is performed to read the historical medical data 211 from the memory unit 200. The model training step S04 is performed to train the historical medical data 211 according to the machine learning algorithm 230 to generate a bloodstream infection prediction model 310. The second data reading step S06 is performed to read the real-time data to be tested 221 of the patient from the memory unit 200. The risk predicting step S08 is performed to input the real-time data to be tested 221 into the bloodstream infection prediction model 310 to generate the bloodstream infection risk probability 320. Thus, the bloodstream infection predicting system 100 of the present disclosure can collect the feature parameters, which are highly correlated with the bloodstream infection, detected in the ICU to predict the bloodstream infection risk probability 320 of a patient in a specific time interval T23 (as shown in FIG. 4), thereby giving a medical treatment to the patient before the blood culture is completed. The first data reading step S02, the model training step S04, the second data reading step S06 and the risk predicting step S08 are described in more detail below.

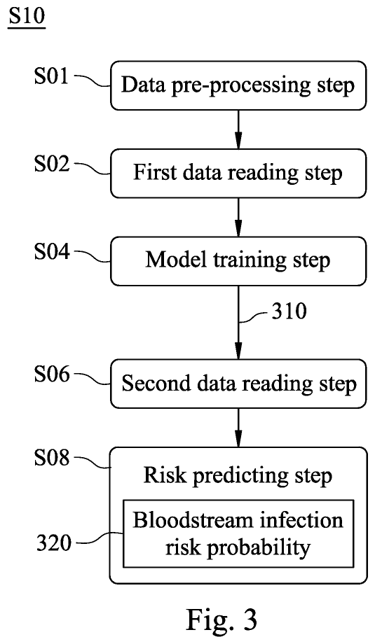

Please refer to FIG. 1 to FIG. 4. FIG. 3 shows a flow chart of a bloodstream infection predicting method S10 according to a second embodiment of the present disclosure. FIG. 4 shows a schematic view of a feature window time interval T12 of the bloodstream infection predicting method S10 of FIG. 3. The bloodstream infection predicting method S10 is configured to predict a bloodstream infection risk probability 320 according to a real-time data to be tested 221 of a patient. The bloodstream infection predicting method S10 includes performing a first data reading step S02, a model training step S04, a second data reading step S06 and a risk predicting step S08.

In FIG. 4, a time point t0 of the time axis t represents a time point when the patient is admitted to the ICU. A time period between the time point t1 and the time point t2 is the feature window time interval T12. A time period between the time point t2 and the time point t3 is a specific time interval T23.

The first data reading step S02 is performed to configure the processor 300 to read the historical medical data 211 from the memory unit 200.

The model training step S04 is performed to configure the processor 300 to train the historical medical data 211 according to the machine learning algorithm 230 to generate the bloodstream infection prediction model 310. Moreover, the machine learning algorithm 230 can be one of a logistic regression, a Support Vector Machine (SVM), a MultiLayer Perceptron (MLP), a random forest and an eXtreme Gradient Boosting (XGBoost), but the present disclosure is not limited thereto.

The second data reading step S06 is performed to configure the processor 300 to read the real-time data to be tested 221 of the patient from the memory unit 200. In detail, the real-time data to be tested 221 read from the real-time database 220 is the real-time data to be tested 221 of a patient admitted to the ICU, and the real-time data to be tested 221 is read during the feature window time interval T12. In the embodiment of FIG. 3, the second data reading step S06 is performed to read the real-time data to be tested 221 of the patient at the time point t2, and the real-time data to be tested 221 is detected between the time point t1 and the time point t2, and the feature window time interval T12 is 72 hours. In other words, the second data reading step S06 reads all the real-time data to be tested 221 of the patient from 72 hours (the time point t1) ago to the present time point t2, but the present disclosure is not limited thereto.

The risk predicting step S08 is performed to configure the processor 300 to input the real-time data to be tested 221 into the bloodstream infection prediction model 310 to generate the bloodstream infection risk probability 320. The real-time data to be tested 221 includes the ICU detecting data 2211 and the blood inspection data 2212 of the patient. The ICU detecting data 2211 and the blood inspection data 2212 are detected during the feature window time interval T12. Furthermore, the bloodstream infection prediction model 310 is configured to predict the bloodstream infection risk probability 320 of the patient at the time point t3. In the embodiment of FIG. 3, the specific time interval T23 between the time point t3 and the time point t2 is 24 hours, but the present disclosure is not limited thereto.

Thus, the bloodstream infection predicting method S10 of the present disclosure reads the real-time data to be tested 221 of the patient during the feature window time interval T12 constantly via the second data reading step S06, and predicts the bloodstream infection risk probability 320 of the patient after the specific time interval T23, thereby generating a warning alert immediately, so that medical staff in the ICU can provide a medical treatment immediately and accurately to a bloodstream infection patient.

The bloodstream infection predicting method S10 can further include performing a data pre-processing step S01. Each of the historical medical data 211 includes a plurality of feature data. The memory unit 200 stores a predetermined number and a predetermined lower limit number. The data pre-processing step S01 includes configuring the processor 300 to calculate an average value of each of the feature data, and configuring the processor 300 to judge whether a number of the feature data of each of the historical medical data 211 is less than or equal to the predetermined lower limit number.

In detail, the feature data of each of the historical medical data 211 are corresponding to the ICU detecting data 2211 and the blood inspection data 2212 of the real-time data to be tested 221. The data pre-processing step S01 calculates the average value of each of the feature data of all the historical medical data 211.

In response to determining that the number of the feature data of one of the historical medical data 211 is less than or equal to the predetermined lower limit number, the processor 300 removes the one of the historical medical data 211. In other words, the data pre-processing step S01 is configured to verify whether the feature data of each of the historical medical data 211 is missing, and removes the historical medical data 211 from a training set of the bloodstream infection prediction model 310 when the number of the missing feature data is more than the predetermined lower limit number, that is, the aforementioned historical medical data 211 will not be a training sample of the bloodstream infection prediction model 310. In the embodiment of FIG. 3, in response to determining that the feature data of each of the historical medical data 211 is complete, the number of the feature data is equal to the predetermined number, and the number of the feature data of each of the historical medical data 211 is 20, the predetermined lower limit number can be 60% of the number of the feature data (i.e., 12 feature data). For example, in response to determining that the number of the missing part of the feature data of one historical medical data 211 is more than 40% of the number of the feature data (i.e., 8 feature data), the one historical medical data 211 will be removed, but the present disclosure is not limited thereto.

In response to determining that the number of the feature data of the one of the historical medical data 211 is greater than the predetermined lower limit number and less than the predetermined number, the processor 300 fills the average values corresponding to a missing part of the feature data of the one of the historical medical data 211 in the one of the historical medical data 211 according to an interpolation process to let the number of the feature data of the one of the historical medical data 211 be equal to the predetermined number. In detail, in response to determining that a small part of the feature data of the one of the historical medical data 211 are missing, the data pre-processing step S01 fills the average values corresponding to the missing feature data into the one of the historical medical data 211 to train the bloodstream infection prediction model 310. In the embodiment of FIG. 3, in response to determining that one of the feature data of one of the historical medical data 211 is missing, the data pre-processing step S01 calculates an average value of the aforementioned one of the feature data of all the historical medical data 211, and fills the average value of the missing feature data into the one of the historical medical data 211.

Thus, the bloodstream infection predicting method S10 of the present disclosure can filter out the incomplete historical medical data 211, decrease deviation of the predicting value of the bloodstream infection prediction model 310, and increase the accuracy of the bloodstream infection risk probability 320, thereby decreasing the bloodstream infection rate and increasing the health care quality to cut down the inpatient days of the ICU.

According to the aforementioned embodiments and examples, the advantages of the present disclosure are described as follows. 1. The bloodstream infection predicting system of the present disclosure can collect the feature parameters, which are highly correlated with the bloodstream infection, detected in the ICU to predict the bloodstream infection risk probability of a patient in a specific time interval, thereby giving a medical treatment to the patient before the blood culture is completed. 2. The bloodstream infection predicting method of the present disclosure reads the real-time data to be tested of the patient during the feature window time interval constantly via the second data reading step, and predicts the bloodstream infection risk probability of the patient after the specific time interval, thereby generating a warning alert immediately, so that medical staff in the ICU can provide a medical treatment immediately and accurately. 3. The bloodstream infection predicting method of the present disclosure can filter out the incomplete historical medical data, decrease deviation of the predicting value of the bloodstream infection prediction model, and increase the accuracy of the bloodstream infection risk probability, thereby decreasing the bloodstream infection rate and increasing the health care quality to cut down the inpatient days of the ICU.

Although the present disclosure has been described in considerable detail with reference to certain embodiments thereof, other embodiments are possible. Therefore, the spirit and scope of the appended claims should not be limited to the description of the embodiments contained herein. In view of the foregoing, it is intended that the present disclosure cover modifications and variations of this disclosure provided they fall within the scope of the following claims.

What is claimed is:

1. A bloodstream infection predicting system, which is configured to predict a bloodstream infection risk probability according to a real-time data to be tested of a patient, and the bloodstream infection predicting system comprising:
a memory unit storing a plurality of historical medical data, the real-time data to be tested and a machine learning algorithm; and
a processor signally connected to the memory unit, and configured to implement a bloodstream infection predicting method comprising:
performing a first data reading step to read the historical medical data from the memory unit;
performing a model training step to train the historical medical data according to the machine learning algorithm to generate a bloodstream infection prediction model;
performing a second data reading step to read the real-time data to be tested of the patient from the memory unit; and
performing a risk predicting step to input the real-time data to be tested into the bloodstream infection prediction model to generate the bloodstream infection risk probability;
wherein the real-time data to be tested comprises an intensive care unit detecting data and a blood inspection data of the patient, and the intensive care unit detecting data and the blood inspection data are detected during a feature window time interval.

2. The bloodstream infection predicting system of claim 1, wherein the memory unit stores a predetermined number and a predetermined lower limit number, each of the historical medical data comprises a plurality of feature data, and the bloodstream infection predicting method further comprises:
performing a data pre-processing step comprising:
configuring the processor to calculate an average value of each of the feature data; and
configuring the processor to judge whether a number of the feature data of each of the historical medical data is less than or equal to the predetermined lower limit number;
wherein in response to determining that the number of the feature data of one of the historical medical data is less than or equal to the predetermined lower limit number, the processor removes the one of the historical medical data; and
wherein in response to determining that the number of the feature data of the one of the historical medical data is greater than the predetermined lower limit number and less than the predetermined number, the processor fills the average values corresponding to a missing part of the feature data of the one of the historical medical data in the one of the historical medical data according to an interpolation process to let the number of the feature data of the one of the historical medical data be equal to the predetermined number.

3. The bloodstream infection predicting system of claim 1, wherein the intensive care unit detecting data comprises a temperature, a respiration rate, a pulse rate, a pulse pressure, a Systolic Blood Pressure (SBP), a Diastolic Blood Pressure (DBP), a Glasgow Coma Scale (GCS) and a catheter insertion time data.

4. The bloodstream infection predicting system of claim 1, wherein the blood inspection data comprises a lactate, an arterial blood gas_pH and a HCO₃-A value.

5. The bloodstream infection predicting system of claim 1, wherein the machine learning algorithm is one of a logistic regression, a Support Vector Machine (SVM), a MultiLayer Perceptron (MLP), a random forest and an eXtreme Gradient Boosting (XGBoost).

6. A bloodstream infection predicting method, which is configured to predict a bloodstream infection risk probability according to a real-time data to be tested of a patient, and the bloodstream infection predicting method comprising:
performing a first data reading step to configure a processor to read a plurality of historical medical data from a memory unit;
performing a model training step to configure the processor to train the historical medical data according to a machine learning algorithm to generate a bloodstream infection prediction model;
performing a second data reading step to configure the processor to read the real-time data to be tested of the patient from the memory unit; and
performing a risk predicting step to configure the processor to input the real-time data to be tested into the bloodstream infection prediction model to generate the bloodstream infection risk probability;
wherein the real-time data to be tested comprises an intensive care unit detecting data and a blood inspection data of the patient, and the intensive care unit detecting data and the blood inspection data are detected during a feature window time interval.

7. The bloodstream infection predicting method of claim 6, wherein the memory unit stores a predetermined number and a predetermined lower limit number, each of the historical medical data comprises a plurality of feature data, and the bloodstream infection predicting method further comprise:
performing a data pre-processing step comprising:
configuring the processor to calculate an average value of each of the feature data; and
configuring the processor to judge whether a number of the feature data of each of the historical medical data is less than or equal to the predetermined lower limit number;
wherein in response to determining that the number of the feature data of one of the historical medical data is less than or equal to the predetermined lower limit number, the processor removes the one of the historical medical data; and
wherein in response to determining that the number of the feature data of the one of the historical medical data is greater than the predetermined lower limit number and less than the predetermined number, the processor fills the average values corresponding to a missing part of the feature data of the one of the historical medical data in the one of the historical medical data according to an interpolation process to let the number of the feature data of the one of the historical medical data be equal to the predetermined number.

8. The bloodstream infection predicting method of claim 6, wherein the intensive care unit detecting data comprises a temperature, a respiration rate, a pulse rate, a pulse pressure, a Systolic Blood Pressure (SBP), a Diastolic Blood Pressure (DBP), a Glasgow Coma Scale (GCS) and a catheter insertion time data.

9. The bloodstream infection predicting method of claim 6, wherein the blood inspection data comprises a lactate, an arterial blood gas_pH and a HCO₃-A value.

10. The bloodstream infection predicting method of claim 6, wherein the machine learning algorithm is one of a logistic regression, a Support Vector Machine (SVM), a MultiLayer Perceptron (MLP), a random forest and an eXtreme Gradient Boosting (XGBoost).
